(19) United States
(12) Patent Application Publication
(10) Pub. No.: US 2003/0022926 A1
(43) Pub. Date: Jan. 30, 2003

(54) Method for treating neuropathic pain and pharmaceutical preparation therefor

(76) Inventor: Patricia Lavand'Homme, Brussel (BE)

Correspondence Address: KNOBBE MARTENS OLSON & BEAR LLP, 2040 MAIN STREET, FOURTEENTH FLOOR, IRVINE, CA, 92614, US

(21) Appl. No.: 10/141,532

(22) Filed: May 7, 2002

Related U.S. Application Data
(60) Provisional application No. 60/289,063, filed on May 7, 2001.

(51) Int. Cl. A61K 31/4168; A61M 5/00

(52) U.S. Cl. 514/388; 604/187

(57) ABSTRACT

The present invention relates to a method for a sustained treatment and/or prophylaxis of neuropathic pain in mammal comprising administering by peripheral nerve injection a neuropathic pain relieving composition comprising an alpha-2-adrenergic agonist.

The invention further relates to the use of an alpha-2-adrenergic agonist for the preparation of an injectable medicament for the sustained treatment and/or prophylaxis of neuropathic pain in mammal by peripheral nerve block.

### TECHNICAL FIELD OF THE INVENTION

This invention relates to the relief and/or prophylaxis of peripheral neuropathic pain syndromes and to improved pharmaceuticals therefor.

### BACKGROUND OF THE INVENTION

Neuropathic pain is a category of pain, which includes several forms of non-nociceptive chronic pain, which result from dysfunction of nervous rather than somatic tissue. The majority of non-nociceptive chronic pains, in terms of either syndromes or cases, follow at various times after damage to either central or peripheral nervous tissue. Diagnosis of most of these syndromes and cases reveals a dependence on abnormal spatial and temporal summation of natural somatic stimulation in the spinal cord and independence from somatic disease and peripheral sympathetic nervous system activity. The scientific pain research community defines this kind of pain as centrally mediated neuropathic pain and recognizes mechanistic, diagnostic, and therapeutic commonalties among pains of this class and differences between these and other syndromes. Neuropathic pain can be defined as pain deriving from damage to or inflammation of central or peripheral nervous systems. Examples of pain syndromes of this class include post herpetic neuralgia, neuritis, temporomandibular disorder, myofascial pain, back pain, pain induced by inflammatory conditions. Neuropathic pain may occur in all body regions. Thus, neuropathic pain may e.g. originate from dental region. Burn injury also often leads to neuropathic hyperalgesia in the affected body area. Neuralgia is characterized, in its acute phase, by intraneural inflammation which can cause damage to primary afferent axons, thus inducing neuropathic pain. Neuropathic pain may also be induced by diabetic conditions (diabetic neuropathy). Neuropathy of primary afferent axons in long nerves is found in diabetic patients. Nociceptor sensitization may ensue. The state of the art concerning peripheral neuropathic pain, common features and clinical treatments (especially new spinal drugs devoid of opioids-related liabilities) has been summarized in several articles (Pain 1997; 73: 123-139; Anesthesiology 1999; 91: 1891-1918).

Chronic pain resulting from peripheral nerve injury (consecutive to cancer invasion, surgery, radiotherapy, chemotherapy, trauma...) is not uncommon in clinical practice. Such injury provokes spontaneous pain, hyperesthesia (enhanced sensitivity to a natural stimulus), hyperalgesia (abnormal sensitivity to noxious stimulus), allodynia (widespread tenderness, characterized by hypersensitivity to non-noxious stimulus) . . . all features which are particularly distressing for the patient and difficult to relieve with classical drugs. Neuropathic pain is reported to be “opioid-poorly-responsive-pain ”.

In some cases, when oral medications fail, the use of spinal drugs represents the last alternative to alleviate pain. However, spinal route is an invasive therapy which is not devoid of dangerous side effects both related to the technique (technical complications, leaks, haematoma, central nervous system infection . . . ) and to the spinally administered drugs. Very few spinal substances are available for human use today: the risks of neurotoxicity and a small therapeutic ratio (i.e. discrepancy between analgesic effect and side effects) are the main reasons. For example, spinal morphine displays poor efficacy in some neuropathic pain conditions and a long lasting use produces many bothersome side effects as well as opioids-related liabilities.

In contrast with the relative lack of effectiveness of opioids, alpha-2-adrenergic agonists are used with success to relieve chronic neuropathic pain, both in animal models and in humans. Further, they represent an attractive alternative to currently used analgesics because they are devoid of respiratory depressant effect and addictive liability.

Alpha-2-adrenergic agonists are drugs commonly used in medical practice as antihypertensive substance and in clinical anesthesiology as component of general and locoregional anesthesia and analgesia. They produce anxiolysis, analgesia, sedation, anesthetic sparing effects and peri-operative hemodynamic stabilizing effects. Negative neurotoxicity studies allow their use (mainly clonidine) by systemic and perimedullar routes and for peripheral nerve blocks. . Among the clinically available alpha-2-adrenoreceptors agonists, clonidine remains widely used: the substance is devoid of neurotoxicity and displays less side effects (i.e. hypotension and sedation) than the more potent and also more alpha-2-selective agonist, dexmedetomidine. Clonidine, a potent alpha-2-adrenergic partial agonist, was used primarily for the treatment of hypertension. This drug stimulates alpha-2-adrenoceptors in the vasomotor centers, causing a reduction of sympathetic outflow from the central nervous system. Both cardiac output and peripheral resistance are reduced resulting in a decrease in blood pressure. Higher concentrations cause a vasoconstriction by activation of postsynaptic receptors in vascular smooth muscle. However, the significant advantages of the drug are counter balanced by side effects including dryness of the mouth, sedation and dizziness.

Besides the well-known analgesic effects of spinally administered alpha-2-adrenergic agonists, their peripheral use has been commonly reported in acute pain conditions. For peri-operative analgesic techniques, clonidine is added to local anesthetic in peritroncular nerve blocks to enhance potency and duration of analgesia (Murphy DR “A non-surgical approach to low back pain” Med. Health R. I., 2000 April; 83(4): 104-7). Further, intra-articular injection of clonidine and its adjunction to local anesthetic solution for intravenous regional anesthesia have also displayed anti-nociceptive effect (Gentili M, Juhel A, Bonnet F. Peripheral analgesic effect of intra-articular clonidine. Pain 1996; 64: 593-596; Reuben S, Steinberg R, Klatt J et al. Intravenous regional anesthesia using Lidocaine and Clonidine. Anesthesiol. 1999; 91: 654-658).

Clonidine, represents an alternative to spinal morphine to relieve neuropathic pain but its spinal use (by intrathecal or epidural route) is strongly limited by side effects such as hypotension and sedation. Systemic clonidine as well, provides minimal analgesia in patients, partly because systemic use of the drug is limited by bothersome dose-related side effects In consequence, any effective alternative to spinal route deserves attention, especially whether the procedure and the injected drug do not produce major side effects which limit the quality of life of the patient.

It is an object of the invention to provide compounds with neuropathic pain alleviating activity and to develop new pharmaceutical preparations suitable for the treatment and/or prophylaxis of neuropathic pain, having a favorable therapeutic ratio with only low side effects.

Another object of the invention relates to the injection of an alpha-2-adrenergic agonist (e.g. clonidine) close to the injured nerve using a peripheral nerve block technique. Such a technique is well known in anesthesiology practice where clonidine is commonly added to local anesthetics to relieve postoperative acute pain—clonidine alone usually does not display peripheral analgesic properties in acute pain conditions.

### SUMMARY OF THE INVENTION

The invention pertains to a method for the sustained treatment and/or prophylaxis of neuropathic pain in mammal comprising administering by peripheral nerve injection a sufficient amount of an effective neuropathic pain relieving composition comprising an alpha-2-adrenergic agonist. The invention further relates to pharmaceutically acceptable salts thereof, and optionally an anaesthetic and/or at least one of a pharmaceutical excipient or an additive. Preferably said alpha-2-adrenergic agonist is clonidine.

Peripheral nerve block is a percutaneous injection of a peripheral nerve (i.e. outside the spinal canal—spinal injection corresponding to a central or neuraxial block). Peripheral nerve injection thus concerns the spinal nerve roots, the peripheral nerves, the plexus and the neuromas. Peripheral nerve block is usually performed by percutaneous injection, with or without a nerve stimulator to find and approach the nervous structure.

In medical literature, the use of peripheral nerve block to relieve chronic neuropathic pain is mainly limited to neuroma infiltration with local anesthetics and steroids mixture, and to neurolytic blocks. Neuroma infiltration brings conflicting results and neurolytic blocks lead to major side effects (J Pain Symptom Manage 1996; 11: 181-187; The Clinical Journal of Pain 2000; 16: S56-S61). Peripheral troncular nerve blocks are sparsely mentioned to relieve untractable chronic pain; in cancer patients, continuous local anesthetic infusions via femoral or axillary catheter have been used with side effects like motor blockade and potential systemic toxicity related to the infused doses.

Peripheral use of an alpha-2-adrenergic agonist in neuropathic conditions has been reported with application of transdermal clonidine—clonidine patches—but the efficacy is limited. Transdermal clonidine provides minimal analgesia in patients with diabetic neuropathy (Pain 1992; 48: 403-408) and reduces sympathetically maintained pain (Anesthesiology 1992; 76: 1065-1066). Borgman in U.S. Pat. No. 6,447,947; describes sympathetically maintained peripheral neuropathic pain relief by topically applying clonidine in an aqueous gel. Topical effect was reported to be inconstant and low.

These and other objects and features of the invention will become more fully apparent when the following detailed description of the invention is read in conjunction with the accompanying examples.

### BRIEF DESCRIPTION OF THE FIGURES AND TABLES

FIG. 1 shows a comparison between the antiallodynic efficacy of intramuscular (IM) clonidine and clonidine sciatic block (SB) in rats.

FIG. 2 shows the effect of clonidine sciatic block (SB) on mechanical allodynia in rats, expressed as % MPE (Maximum Possible Effect).

FIG. 3 shows the effect of clonidine sciatic block (SB) on thermal hyperalgesia, expressed as Δ PWL (Paw Withdrawal Value) value between pre-ligation and post-ligation values

Table 1 shows PWT values (Paw Withdrawal threshold) after sciatic nerve block with Saline and intramuscular injection with clonidine 30 μg.

Table 2 shows PWT values (Paw Withdrawal threshold) after peripheral sciatic nerve block with clonidine 3, 15, 30 μg.

### DETAILED DESCRIPTION OF THE INVENTION

The present invention relates to a method for a sustained treatment and/or prophylaxis of neuropathic pain in mammal comprising administering by peripheral nerve injection a neuropathic pain relieving composition comprising an alpha-2-adrenergic agonist. In a preferred embodiment the alpha-2-adrenergic agonist is clonidine or dexmedetomidine.

The present invention further relates to the use of an alpha-2-adrenergic agonist for the preparation of an injectable medicament for the sustained treatment and/or prophylaxis of neuropathic pain in mammal by peripheral nerve block. In a preferred embodiment the alpha-2-adrenergic agonist is clonidine or dexmedetomidine.

It is an aim of the present invention to further provide pharmaceutical injectable compositions for the sustained treatment and/or prophylaxis of neuropathic pain in mammal by peripheral nerve injection, comprising essentially an alpha-2-adrenergic agonist and optionally an anaesthetic and/or at least one of a pharmaceutical excipient or an additive. The present invention further relates to a method for a sustained treatment and/or prophylaxis of neuropathic pain in mammal wherein the pharmaceutical injectable compositions is injected in the periphery of, preferably in a position as close as possible to the injured nerves.

Alpha-2-adrenergic agonists, e.g. clonidine and dexmedetomidine, are non-neurotoxic substance which displays higher efficacy in neuropathic pain conditions, they have the advantage to be devoid of most of the major side effects which result from chronic administration of the classical substances used to relieve neuropathic pain. In contrast with local anesthetics, motor blockade, cardiovascular or central nervous system toxicity, have not been reported as well as well known side effects consecutive to steroids use are usually not observed after alpha-2-adrenergic agonists administration.

According to the invention, alpha-2-adrenergic agonists are used for the preparation of medicaments for the sustained treatment and/or prophylaxis of neuropathic pain in mammal by peripheral nerve block.

The term “peripheral nerve block” as used herein refers to a percutaneous injection of a peripheral nerve (i.e. outside the spinal canal—spinal injection corresponding to a central or neuraxial block). Peripheral nerve block is usually performed by percutaneous injection, with or without a nerve stimulator to find and approach the nervous structure. Peripheral nerve injection thus concerns the spinal roots, the peripheral nerves, the plexus and the neuromas

The main advantages to peripheral nerve blocks are to selectively restrict the analgesic effect to one sensitive territory and to allow loco-regional analgesia in patients for whom the coagulation parameters are not optimal. Furthermore, the perineural injection of alpha-2-adrenergic agonist is easily realizable and provides long lasting effect. In consequence, the problems related to placement of an invasive drug delivery system can be strongly minimized. Health-related quality of life, patient satisfaction and economic assessment might be improved with such a treatment, especially in chronic pain conditions.

According to the method of the invention, an injection of clonidine induces a long lasting pain relief, both in human case and in an animal model of neuropathic pain. The method is relatively safe, devoid of major drug's side effects and allows chronic pain relief without the use of too invasive technique.

The invention relates also to pharmaceutical injectable compositions for the sustained treatment and/or prophylaxis of neuropathic pain in mammal by peripheral nerve injection, comprising essentially an alpha-2-adrenergic agonist and optionally an anaesthetic and/or at least one of a pharmaceutical excipient or an additive.

The compounds used in accordance with the invention are known. Pharmaceutical preparations containing clonidine are commercially available as antihypertensive medications under the trade name Catapressan® (Boehringer Ingelheim). The compounds can be manufactured in a known manner essentially in accordance with the processes described in the prior arts. According to the invention, alpha-2-adrenergic agonist such as clonidine and its physiologically compatible acid-addition salts are used for the manufacture of pharmaceutical preparations for the treatment and/or prophylaxis of neuropathic pain.

The alpha-2-adrenergic agonist of the invention may be clonidine, p-aminoclionidine, tiamenidine, 5-bromo-6-(2 imidazolidine-2-ylamino) quinoxaline, dexmedetomidine, detomidine, medetomidine, oxymetazonline, brimonidine, tizanidine, mivazerol (UCB-Pharma, Belgium), lofexidine, xylazine, guanabenz, guanfacine, guanclofine, guanoxabenz, or a derivative or structural analogue thereof, alpha-methyinorepherine, azepexole, indoramin, 6-allyl-2-amino-5,6,7,8-tetrahydro4H-thiazolo [4,5-d]azepine diHCl and analogs thereof

The term “clonidine” as used herein refers to N-(2,6-dichlorophenyl)-4,5-dihydro-1H-imidazol-2-amine and includes the pharmaceutically acceptable salts thereof, e.g., salts with inorganic acids, such as hydrohalic acids, or with organic acids, for example lower aliphatic monocarboxylic or dicarboxylic acids such as acetic acid, fumaric acid or tartaric acid or aromatic carboxylic acids such as salicylic acid are also suitable.

Clonidine is employed in a therapeutically effective amount, preferably in the form of clonidine base. The actual concentration of clonidine may vary, depending on the nature and degree of the pain syndromes being treated and whether the drug is being administered for therapeutic or prophylactic purposes.

Preservatives may be incorporated in an amount effective for inhibiting growth of microbes, such as bacteria, yeast and molds, in the composition. Any conventional preservative against microbial contamination of the product can be employed so long as it is pharmaceutically acceptable and is unreactive with clonidine. Preferred preservatives are antimicrobial aromatic alcohols, such as benzyl alcohol, phenoxyethanol, phenethyl alcohol, and the like, and esters of parahydroxybenzoic acid commonly referred to as paraben compounds, such as methyl, ethyl, propyl, and butyl esters of parahydroxybenzoic acid and the like and mixtures thereof, but are not limited thereto. Particularly preferred are benzyl alcohol and phenoxyethanol.

Optionally, anaesthetic agent, such as lidocaine and the like, can be included. For administration according to the invention the active quantities of the compounds that alleviate neuropathic pain can be contained together with customary pharmaceutical excipients and/or additives in solid or liquid pharmaceutical formulations.

Liquid preparations such as solutions, suspensions or emulsions of the active ingredients can contain the usual diluents such as water, oil and/or suspending aids such as polyethylene glycols and such like. Further additives such as preservatives, flavoring agents and such like may also be added.

The active ingredients can be mixed and formulated with the pharmaceutical excipients and/or additives in a known manner. For the manufacture of solid dosage forms, for example, the active ingredients may be mixed with the excipients and/or additives and granulated in a wet or dry process. Granules or powder can be filled directly into capsules or compressed into tablet cores. If desired, these can be coated in the known manner.

Liquid preparations can be prepared by dissolving or dispersing the compounds and optional pharmaceutical adjuvants, in a carrier, such as, for example, aqueous saline, aqueous dextrose, glycerol, or ethanol, to form a solution or suspension.

Desirably, the composition of this invention is injected to the affected painful region of a patient to deliver targeted high concentrations to the painful site yet afford minimum systemic concentrations.

The present invention also relates to the use of alpha-2-adrenergic agonist for the manufacture of a device for delivering to a peripheral nerve or a close region thereof, preferably to an injured nerve, a therapeutic dose of said agonist by peripheral nerve injection said device comprising reservoir means for storing said agonist, a therapeutic dosage form of said agonist disposed in said reservoir means, a piston means movable along the reservoir means along a longitudinal axis for dispensing said therapeutic dosage form of said agonist to a hollow needle mounted on said reservoir means for delivering said agonist to the peripheral nerve of a patient. In a preferred embodiment, the alpha-2-adrenergic agonist is clonidine or dexmedetomidine. Said alpha-2-adrenergic agonist, can be for example formulated as an injectable solution. Said device can be for example a syringe. The hollow needle can be manufactured to prevent, reduce or eliminate trauma to the nerve during the injection of said alpha-2-adrenergic agonist.

The term “injured nerve” as used herein refers to damaged nerve that becomes a cause inducing the neuropathic pain. The damages typically includes traumas and injuries inflicted to the peripheral nervous system, nerve plexus, and soft tissues surrounding the nerves, as well as injuries to the somatesthesia paths in the central nervous system (such as ascending somatesthesia paths found at the levels of spinal cord, brain stem, thalamus and cerebral cortex). Included herein injured nerves can be found in association with nerve degenerative diseases, bone degenerative diseases, metabolic diseases, cancer, infection, inflammation, post-surgery state, trauma, radiation therapy and anti-cancer chemotherapy, etc.

The term “patient” as used herein refers to an animal, preferably a mammal, and most preferably a human, who has been the object of treatment, observation or experiment.

The term “therapeutic dose ” as used herein refers to the amount of alpha-2-adrenergic agonist effective to reduce or eliminate the neuropathic pain.

The present invention also encompasses the use of alpha-2-adrenergic agonist for the manufacture of an injectable solution for the delivery to a peripheral nerve or a close region thereof, preferably to an injured nerve, a therapeutic dose of said agonist by peripheral nerve injection, wherein said solution comprises from 3 μg to 1 mg of said alpha-2-adrenergic agonist. Preferably, said solution comprises from 30 μg to 500 μg of said alpha-2-adrenergic agonist. More preferably said solution comprises from 30 μg to 250 μg of said alpha-2-adrenergic agonist. Yet more preferably said solution comprises from 30 μg to 150 μg of said alpha-2-adrenergic agonist. In a preferred embodiment, the alpha-2-adrenergic agonist is clonidine or dexmedetomidine.

Furthermore, said alpha-2-adrenergic agonist may be used to prevent or treat mechanical allodynia and/or hyperalgesia, for manufacturing an injectable medication for the administration of from 3 μg to 1 mg of alpha-2-adrenergic agonist as a means for treating neuropathic pain. Preferably, said injectable medication provides for the administration of from 30 μg to 500 μg of said alpha-2-adrenergic agonist, more preferably for the administration of from 30 μg to 250 μg of said alpha-2-adrenergic agonist, yet more preferably for the administration of from 30 μg to 150 μg of said alpha-2-adrenergic agonist. A non-limiting example of a suitable amount for administration to a human patient is 150 μg. In a preferred embodiment, the alpha-2-adrenergic agonist is clonidine or dexmedetomidine.

It was surprisingly found that a single peripheral nerve injection with an alpha-2-adrenergic agonist resulted into significantly long lasting and dose dependent relief of neuropathic pain. The injected alpha-2-adrenergic agonist produces surprisingly a maximal neuropathic pain relief after 3 to 7 days and pain relief can last for 1 to 5 weeks.

The present invention therefore also relates to a method of treatment of neuropathic pain in a patient in need thereof, comprising administering to a peripheral nerve or a close region thereof, preferably to an injured nerve of said patient, a therapeutic dose of an alpha-2-adrenergic agonist by peripheral nerve injection every 1 to 5 weeks. In a preferred embodiment, the alpha-2-adrenergic agonist is clonidine or dexmedetomidine.

### Experiments and Results

The following example illustrates the present invention as a more detailed illustration of composition suitable for the treatment and/or prophylaxis of neuropathic pain, without, however, limiting the scope of the application. Since modifications of the disclosed embodiments incorporating the spirit and substance of the invention may occur to persons skilled in the art, the invention should be construed to include everything within the scope of the appended claims and equivalents thereof. Those skilled in the art will appreciate that the foregoing dose levels are provided illustratively, and that higher and lower dose levels can be employed without departing from the spirit and scope of the present invention.

Peripheral nerve blocks, mainly with local anesthetics and steroids, can be used to treat neuropathic pain. In both humans and animals, clonidine is highly effective to alleviate neuropathic pain. Further, in animal models, peripheral nerve injury triggers a build up of alpha-2A-adrenergic receptors close to the lesion. The efficacy of peripheral nerve block with clonidine in relieving mechanical allodynia was assessed in rats after a partial ligation of sciatic nerve. Four to six weeks after nerve injury, peripheral sciatic block with clonidine (3 to 30 μg) resulted into significantly long lasting (peak effect at day 7 post injection) and dose dependent relief of mechanical allodynia. Clonidine injection took effect at injury site because systemic clonidine was ineffective. Immunostaining studies confirmed the presence of adrenergic α2A-receptors in the injury site (around the sutures). Peripheral clonidine injection also dramatically suppressed immunostaining for phosphorylated cAMP response element-binding protein (pCREB) in the superficial dorsal horn ipsilateral to injury. These data suggest that partial ligation of sciatic nerve injury up-regulates adrenergic alpha-2A receptors at the injury site and peripherally administered clonidine likely activates these receptors to achieve its anti-allodynic effects, thus suppressing in dorsal horn neurons phosphorylation of CREB which have been up-regulated by noxious signals originating from the injured nerve.

The neuropathic pain alleviating activity of clonidine is demonstrated in rats and human by standard tests for the evaluation of neuropathic pain inhibiting activities. The experimental data found show that clonidine is effective in relieving both thermal hyperlagesia and mechanical allodynia which is a form of neuropathic pain. Thus, the results of the experiments according to the present invention demonstrate the potential for clonidine and other alpha-2-adrenergic compounds for the treatment of neuropathic pain.

Using a validated animal model of neuropathic pain (partial ligation of one sciatic nerve—Seltzer model—Seltzer Z, Dubner R, Shir Y. A novel behavioral model of neuropathic pain disorders produced in rats by partial sciatic nerve injury. Pain 1990; 43: 205-218.), an ipsilateral percutaneous sciatic block injection of clonidine alleviates both mechanical allodynia and thermal hyperalgesia of the injured paw. The injected preparation produces surprisingly a maximal antihyperalgesic effect after 3 to 7 days and pain relief lasts for 10 to 14 days. Very similar results have been observed in a cancerous patient with neuropathic pain, who gets effective and long lasting pain relief after peripheral nerve block with clonidine. Repetitive blocks have been performed until now in this patient without apparition of tolerance to their efficacy.

A male patient, 70 years old, presented neuropathic pain symptoms related to the presence of a right iliac mass which was an hypernephroma metastasis. The patient complained of both spontaneous pain, increasing with movements like walking or standing up, and evoked pain such as mechanical allodynia and hyperalgesia. The pain symptoms were localized to the anterior and exterolateral face of his right thigh and resulted from the compression of the patient's right femoral nerve at the psoas level by the tumor metastasis (CT SCAN). The pain was not responding to orally administered opiates, at least at doses devoid of side effects; increase of analgesic doses provoked hallucinations and nausea and vomiting. The patient was then referred to the Pain Clinic to benefit from neuraxial analgesia (either epidural or intrathecal catheter placement). However, long-term spinal catheter is not devoid of side effects, both related to the technique, especially the risk of infection and to the drugs used. In this case, the topography of the neuropathic pain, perfectly limited to the area of the right femoral nerve, allowed to try a peripheral nerve injection as analgesic technique. After patient's agreement, a right femoral nerve block was realized with the use of a nerve stimulator and a single dose of bupivacaine 15 mg, clonidine 150 μg and methylprednisolone depot 40 mg was injected in a total volume of 15 ml. Two hours after injection, the pain was strongly reduced and the patient was released from the hospital without any side effect. Twenty-four hours after the peripheral nerve injection, the pain had totally disappeared and the patient was able to walk without his stick walk. A moderate daily oral complement with paracetamol and codeine kept the patient pain free for three weeks before that he came back to the hospital to receive an other femoral peripheral nerve block. The same analgesic procedure was repeated once a month for six months with success. Local anesthetic and steroid, which respectively can induce motor deficit and systemic hormonal dysregulation, were withdrawn from the initial mixture and it appeared that clonidine alone was as effective as previous combination. Only the onset of the antihyperalgesic effect was delayed in this case (four to five days) but neuropathic pain, especially the mechanical component, remained strongly alleviated for more than ten days after a single femoral block injection. At this time point, six months later, the patient received a last cure of chemotherapy which reduced the psoas mass and allowed him to get free from his therapeutic infiltrations.

A validated animal model of neuropathic pain was used that results from a partial ligation of one sciatic nerve in rat (Seltzer 1990). Nerve ligation and subsequent edema represents an experimental model for mechanisms operative in both nociceptive nerve pain and pain due to partial deafferentation (Asbury A, Fields H. Pain due to peripheral nerve damage: an hypothesis. Neurology 1984; 34: 1587-1590). This model thereby closely mimics the clinical situation found in several cancerous patients.

After Institutional Animal Care Committee approval, adult male Wistar rats (Hanovre, weight 150 -180 g) underwent partial sciatic nerve ligation of the right sciatic nerve as described by Seltzer et al (Seltzer 1990). Briefly, under halothane anesthesia, the right sciatic nerve was exposed at high-thigh level and liberated from surrounding tissues. One third to one half of the nerve was then tightly ligated with silicon-treated silk suture (Prolene 7-0) before those muscle and skin layers were closed. The animals were allowed to recover for four to six weeks, lag of time sufficient to develop neuropathic pain features.

Mechanical allodynia was assessed by application of calibrated Von Frey filaments (Stoelting, Wood Dale, Ill.), ranging from 1.2 g to 28.8 g.

The animals were placed on a plastic mesh floor into individual boxes. After accommodation to the environment, the Von Frey filaments were applied vertically to the plantar surface of the ligated hind paw and gently bent 3-4 times over approximately 2 sec. if no response was elicited, a larger filament was applied in the same manner. The filaments were applied in increasing order until a brisk withdrawal or paw flinching occurred, that was considered as a positive response. The filament applications were repeated three times, with five to ten minutes interval between testing. The withdrawal threshold was determined as the mean of the three values.

Thermal evoked paw withdrawal threshold was measured using a commercially available device modeled after that described by Hargreaves (Dirig D, Salami A, Rathbun M et al. Characterization of variables defining hind paw withdrawal latency evoked by radiant heat thermal stimuli. J. Neurosci. Methods, 1997; 76: 183-191). The rats were placed in a clear plastic cage on an elevated floor of clear, heat-tempered glass. A radiant heat was focused on the plantar surface of the foot and a timer recorded the paw withdrawal latency (PWL) from the heat noxious stimulus. The amperage delivered to the light source, thereby the intensity of the stimulus was monitored to remain constant and a 20.48-s cutoff was used to limit possible tissular damage. Both paws were tested in alternance with a 10-min time interval between testing. To obtain an average paw withdrawal latency, three to four trials were realized. Because partial sciatic nerve ligation mainly induced postural modification with foot evasion of the ligated paw, for every measurement the observer tried to focus the thermal stimulus on the part of the foot which was in full contact with the glass floor.

Only the animals which had developed satisfactory Mechanical Allodynia and Thermal Hyperalgesia were used to assess the efficacy of peripheral nerve blockade.

The rats were anesthetized with 4% halothane inhaled concentration in 100% oxygen by face mask. An unilateral percutaneous sciatic nerve block was then performed on the right ligated side. A 25-gauge needle was introduced between the greater trochanter and the ischial tuberosity, both landmarks previously localized by palpation, according to the technique reported by Thalhammer (Thalhammer JG, et al Neurologic evaluation of the rat during sciatic nerve block with lidocaine. Anesthesiology. 1995 Apr;82(4):1013-25). Different mixtures of saline and alpha-2-adrenergic agonist were then injected in a total volume of 0.3 ml.

A single sciatic block (SB) injection of the following drugs was realized: Saline (n=6), Saline and intramuscular (IM) clonidine 30 μg (n=11), Clonidine 15 μg (n=8), Clonidine 3 μg (n=9). The drug used in the study for peripheral nerve block was: clonidine marketed for clinical use (Catapressan®, Boehringer Ingelheim). The drug was diluted in isotonic saline.

The animals were tested as previously described for both Mechanical Allodynia and Thermal Hyperalgesia at day0 (5 hours after sciatic nerve injection), day 1, day 3, day 7, day 10, day 14 and day 21 post drug injection.

Statistical analysis used two-ways ANOVA with post-hoc comparisons; p<0.05 was considered significant (Statistica 5.0 -version 97-StatSoft).

For Mechanical Allodynia, Paw withdrawal threshold (PWT) values were converted to percent of maximum possible effect (% MPE), according to the formula:

% MPE=(post-drug infiltration threshold−baseline post-ligation threshold)/(pre-ligation threshold−baseline post-ligation threshold)×100

Values are expressed as mean±SD. The percent maximum possible effect data were used to calculate the respective ED50 values of clonidine and clonidine combinations using linear regression.

Concerning Thermal Hyperalgesia, a difference score (ΔPWL) was calculated by subtracting the paw withdrawal latency of the paw before sciatic ligation (control value) from the ligated paw withdrawal latency. A negative value thereby indicated a shorter withdrawal latency to thermal nociceptive stimulus on the operated paw, or thermal hyperalgesia. The contralateral paw could not be used as a control because contralateral side usually displays mirror image of pain, i.e. abnormal mechanical and thermal values in this model (Seltzer 1990).

Values are expressed as mean±SD. To compare the control and operated paw withdrawal latencies and the difference scores in a group, and to compare drug effect, parametric tests were used.

Peripheral sciatic nerve block with clonidine did not result into motor blockade or subsequent motor deficit in rats. Only mild transitory sedation was observed after clonidine injection.

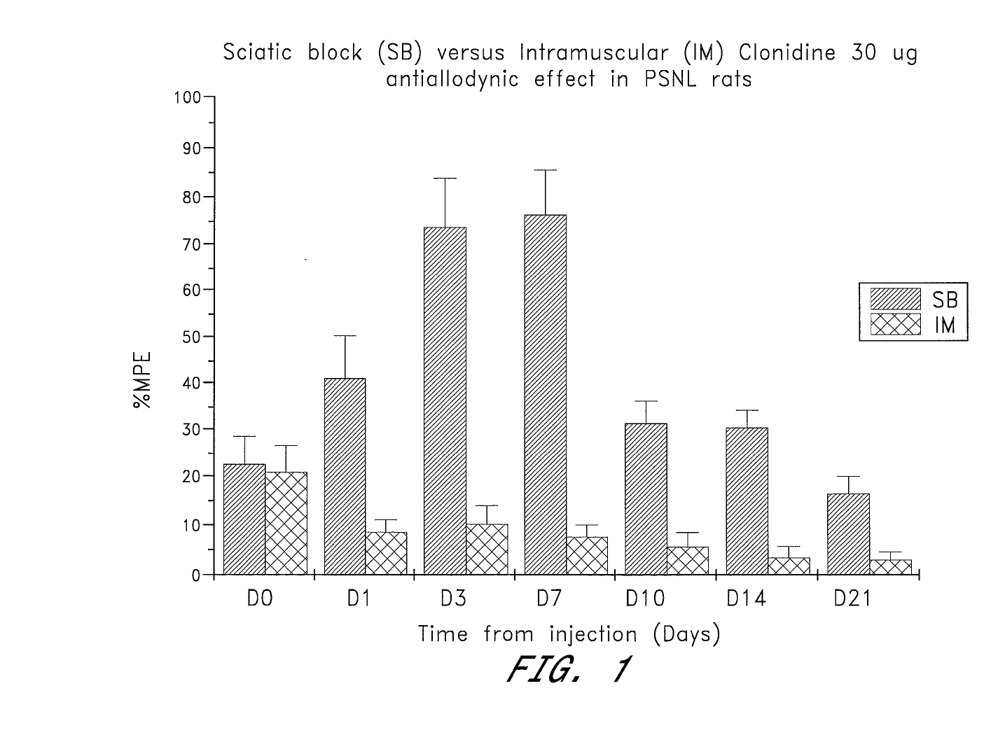

Four to six weeks after partial sciatic nerve ligation, animals developed Mechanical Allodynia with PWT values significantly reduced. Sciatic nerve block with Saline did not influence PWT values as shown in Table 1. Systemic injection (IM) of clonidine 30 μg produced maximal antiallodynic effect of 20.7±5.7 % MPE at day 0 (5 hours post-sciatic blockade) and this effect was non statistically significant. PWT values after IM clonidine are expressed in Table 1. FIG. 1 shows a comparison between the antiallodynic efficacy of IM clonidine 30 μg and SB with clonidine 30 μg. A single peripheral sciatic block with clonidine significantly alleviated mechanical allodynia in neuropathic rats, with potency and duration of effect related to the injected dose. PWT values (in g; * p<0.05 with pre-block value) are related in Table 2 after injection of clonidine 30 μg, 15 μg and 3 μg. Maximal effect of SBCLO 30 μg was 76.6±9.2 % MPE at D7, SBCLO 15μg 40.5±6.4 % MPE at D3 and SBCLO 3 μg 34.2±5.1 % MPE at D1 in alleviating Mechanical Allodynia.

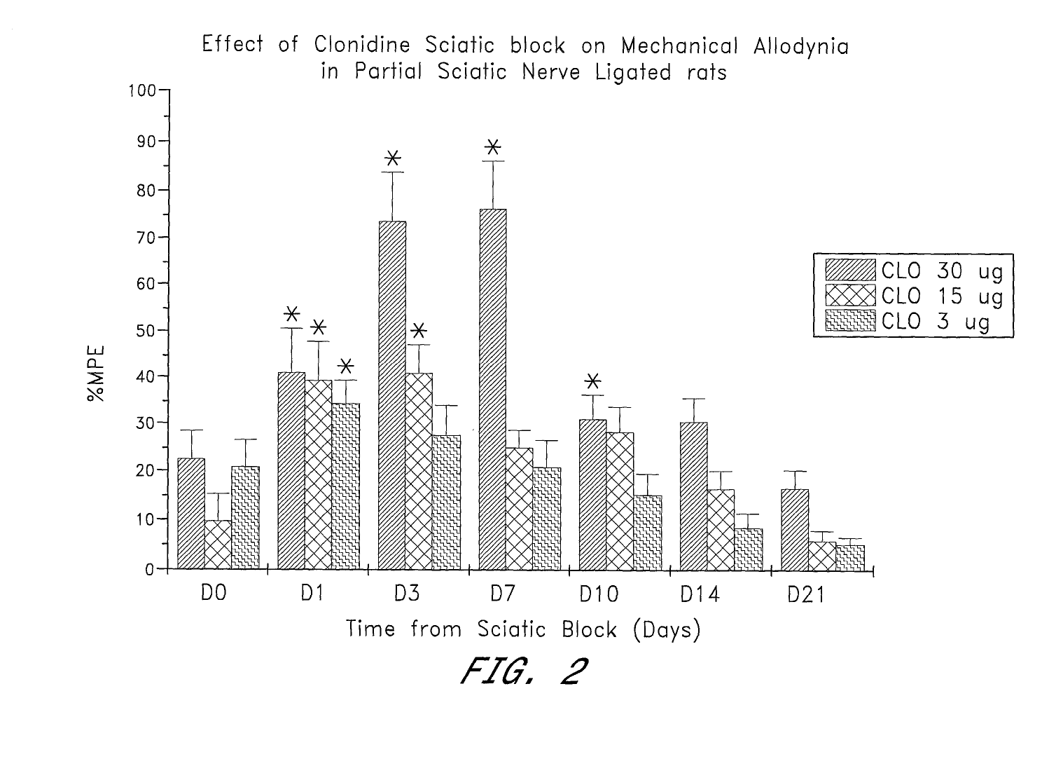

FIG. 2 displays clonidine SB efficacy as neuropathic pain treatment expressed as % MPE to relieve mechanical allodynia (* p<0.05 with pre-block value).

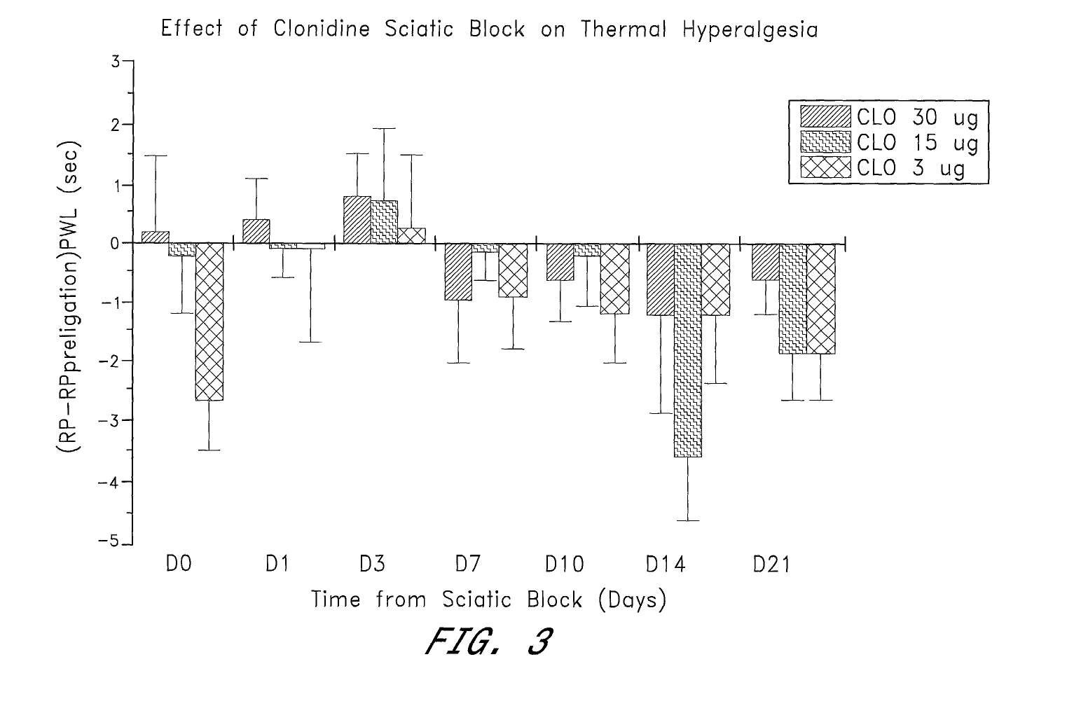

Partial sciatic nerve ligation in rats induced significant thermal hyperalgesia development in the ligated side. Peripheral nerve block with clonidine displayed a long lasting effect and alleviated the thermal hyperalgesia in a dose-related manner. FIG. 3 shows SB clonidine effect on thermal hyperalgesia, expressed as ΔPWL value between pre-ligation and post-ligation values (in sec; * p<0.05 with pre-block value).

The data obtained after animal testing confirmed the beneficial effects observed in the aforementioned human patient. Peripheral nerve block with clonidine alleviates Mechanical Allodynia and Thermal Hyperalgesia in a rat model of peripheral neuropathic pain. Potency and duration of clonidine effect are clearly related to the injected dose like it is the case when clonidine is spinally administered. However, the exceptional long lasting antihyperalgesic effect after peripheral nerve injection deserves to be highlighted and contrasts with short lasting spinal analgesic effect. Such a peripheral injection of clonidine resulted, both in animal model and in human case, into a long lasting antihyperalgesic effect and seemed devoid of major bothersome side effects.

In a further experiment, the effect of peripheral injected clonidine on central plasticity was examined and its effect on the expression of phosphorylated cAMP response element binding protein (PCREB) was studied.

Four normal rats, 4 nerve injured rats and 4 nerve injured rats 1 week after peripheral nerve injection of clonidine, 30 mg were used for quantification. Four weeks after nerve injury and one week after peripheral nerve injection of saline or clonidine, rats were anesthetized with sodium pentobarbital and perfused intracardially with cold phosphate buffered saline (PBS) containing 1% sodium nitrite followed by 4% paraformaldehyde in 0.1 M phosphate buffer (PB, pH 7.4). The L4-5 spinal cord segments and the ipsilateral and contralateral sciatic nerve segments at both proximal and mid-thigh levels were removed and postfixed in the same fixative for 3-6 hours. Then all tissues were transferred to 30% sucrose in 0.1 M PB at 40° C. for cryoprotection. The spinal cord segments were cut on a cryostat at a 40 μm thickness. The free-floating sections were collected in PBS. After being pre-treated with 0.3% H 202 and 10% normal goat serum (NGS, Vector Laboratories, Burlingame, Calif.), sections were incubated for 36 hours at 40° C. in a rabbit polyclonal anti-phospho-CREB antibody (1:1,000, New England Biolabs, Beverly, Mass., U.S.A.) diluted in phosphate buffered saline containing 0.3% Triton-×100 (PBS+T) and 3% NGS. Subsequently, the sections were incubated in biotinylated goat anti-rabbit IgG (Vector) and further processed using Elite Vectastain ABC kit (Vector) according to the instructions of the manufacturer. Between each incubation, sections were washed in PBS+T (15 min twice). Finally, the immunoprecipitates were developed by 3,-3′ diaminobenzedine (DAB) and the chromogen was enhanced by the glucose oxidase-nickel-3,-3′ DAB method. Four sciatic nerve segments, ipsilateral at proximal thigh level (containing ligation), contralateral at proximal thigh level, ipsilateral at mid-thigh level and contralateral at mid-thigh level, were mounted on the same block using O.C.T. mounting media. Sections were cut at 10 μm thickness, thaw mounted on precleaned Superfrost/plus slides (Fisher Scientific, Pittsburgh, Pa., U.S.A.) and kept at −80° C. until used. After pretreatment in 0.1% H2O2 and 10% NGS in 0.1 M Tris buffered saline (TBS), sections were incubated in a polyclonal rabbit antibody directed against a2A adrenergic receptor (Generously provided by Dr. R. Elde, Department of Neuroscience, University of Minnesota, Minneapolis, Minn.). Subsequently, the sections were incubated in biotinylated goat anti-rabbit IgG (Vector) and further processed using Elite Vectastain ABC kit (Vector) according to the instructions of the manufacturer. Finally, the immunoprecipitates were developed by glucose oxidase-nickel-DAB method described above. Between each incubation, sections were washed in TBS containing 0.05%+T (15 min twice).

Images of both ipsilateral and contralateral dorsal horns of nerve injured rats or from the dorsal horn of normal rats were captured at ×250 magnification using a CCD camera. In a given area (235 μm×235 μm), which covered the entire LI to LII in depth and was located in the middle one third of the mediolateral extent of the dorsal horn, the number of pixels occupied by pCREB-IR cells on each side was measured automatically using image analysis software (SigmaScan, Jandel Scientific Inc., San Rafael, Calif., U.S.A.). Values are expressed as mean+SEM. Groups were compared using two-way ANOVA followed by Dunnett's or Fishers LSD test. To compare the mean number of the pixels occupied by pCREB-IR cells between two-side dorsal horn, a paired t-test or one-way ANOVA with Student-Newman-Keuls multiple comparison method was used. P<0.05 was considered significant.

Numerous pCREB-IR cells were observed in the dorsal horn of L4-L6 spinal cord of normal rats. The immunoprecipitates were all localized in the nuclei of these cells. PCRE-BIR cells were concentrated in the superficial laminae. The number of pCREB-IR cells was significantly increased in the ipsilateral dorsal horn compared to the contralateral side four weeks after partial sciatic nerve ligation. One week after a single peripheral nerve injection of clonidine at the injury site, this increase in pCREB-IR cells in the ipsilateral dorsal horn was dramatically suppressed. This single injection of clonidine also markedly reduced the number of pCREB-IR cells in the contralateral dorsal horn. Following a single injection of saline into the injury site of nerve ligated rats, the number of pCREB-IR cells in the ipsilateral dorsal horn remained significantly higher than the contralateral side, similar to nerve ligated rats without injection.

Next a quantitative analysis of pCREB-immunoreactive (IR) cells in the dorsal horn of rats with partial sciatic nerve ligation after a peripheral nerve injection of saline or clonidine was performed. The mean number of pixels in a fixed area occupied by pCREB-IR cells in the ipsilateral dorsal horn was significantly greater than the contralateral side in peripheral nerve saline injected controls (p<0.01). However, the mean optical density of pCREB-IR cells in this fixed area was not significantly different between the ipsilateral and contralateral dorsal horn. Following peripheral nerve injection of clonidine in nerve ligated rats, both the mean number of pixels and the mean optical density in a fixed area occupied by pCREB-IR cells in the dorsal horn were significantly reduced ipsilateral and contralateral to ligation/injection site compared to controls (p<0.001).

Peripheral nerve block with clonidine produces a long lasting and delayed action by affecting immune cell function near the site of nerve injury. Clonidine most likely acts by reducing cytokine and growth factor production by immune cells, perhaps additionally inducing apoptosis in some immune cells, thereby reducing centrally transported cytokines and growth factors and reducing central sensitization. One week following peripheral nerve injection of clonidine, at the time of maximum reduction in hypersensitivity, pCREB expression was diminished in the spinal cord.

In summary, behavioral and immunocytochemical studies in rats with peripheral nerve injury are consistent with a site- and subtype-specific action on newly expressed a2A-adrenoceptors at the site of injury which reduce hypersensitivity. The delayed onset and prolonged duration observed in the patient and in this animal model, accompanied by reduction in a clonidine-induced reduction in a spinal transcription factor, indicate that peripheral nerve block with clonidine probably acts by altering generation of factors released by immune cells and their central trafficking which regulates and maintains hypersensitivity. The present findings show the effectiveness of peripheral nerve injected alphe-2-adrenergic agonists in the treatment of nerve injury-associated pain in humans. The present findings highlight the utility of peripheral nerve block with an alpha-2-adrenergic agonist, e.g. clonidine, to relieve some neuropathic pain conditions in humans.

TABLE 1
PWT values (Paw Withdrawl thresholding) after sciatic nerve block with Saline and
intramuscular injection with clonidine 30 μg.

| SB | | | | | | | |
|---|---|---|---|---|---|---|---|
| injection | D0 (5 h) | D1 | D3 | D7 | D10 | D14 | D21 |
| Saline | 2.0 ± 0.6 | 2.2 ± 0.4 | 2.4 ± 0.4 | 2.2 ± 0.4 | — | — | — |
| IM CLO | 2.6 ± 1.8 | 7.9 ± 4.2 | 4.3 ± 2.8 | 5.2 ± 2.8 | 3.6 ± 2.3 | 2.0 ± 3.3 | 3.6 ± 1.4 |
| 30 μg | | | | | | | |

TABLE 2
PWT values (Paw Withdrawl thresholding) after peripheral sciatic nerve block with
clonidine 3, 15, 30 μg

| SB | | | | | | | |
|---|---|---|---|---|---|---|---|
| injection | D0 (5 h) | D1 | D3 | D7 | D10 | D14 | D21 |
| CLO 30 μg | 9.3 ± 6 | 14.7 ± 10* | 23.7 ± 11* | 23.0 ± 7* | 11.9 ± 3* | 11.4 ± 4 | 7.8 ± 3 |
| CLO 15 μg | 4.8 ± 2 | 12.6 ± 6* | 12.9 ± 5* | 8.8 ± 3 | 9.7 ± 4 | 6.4 ± 3 | 3.7 ± 1 |
| CLO 3 μg | 4.7 ± 4 | 12.0 ± 4* | 10.3 ± 5 | 8.2 ± 4 | 7.0 ± 3 | 5.0 ± 2 | 4.3 ± 1 |

What is claimed is:

1. A method for sustained treatment and/or prophylaxis of neuropathic pain in a mammal comprising:
administering a neuropathic pain relieving composition comprising an alpha-2-adrenergic agonist by peripheral nerve injection.

2. The method of claim 1, wherein said alpha-2-adrenergic agonist is clonidine.

3. The method of claim 1, wherein said alpha-2-adrenergic agonist is dexmedetomidine.

4. A pharmaceutical composition for the sustained treatment and/or prophylaxis of neuropathic pain in a mammal by peripheral nerve injection, comprising an alpha-2-adrenergic agonist, an anaesthetic, and at least one pharmaceutical excipient.

5. The pharmaceutical composition of claim 4, wherein said alpha-2-adrenergic agonist is clonidine.

6. The pharmaceutical composition of claim 4, wherein said alpha-2-adrenergic agonist is dexmedetomidine.

7. A method for sustained treatment and/or prophylaxis of neuropathic pain resulting from injury to a peripheral nerve in a mammal comprising:
administering the pharmaceutical composition of claim 4 by injecting said composition in close proximity to the injured peripheral nerve.

8. A device for delivering a therapeutic dose of an alpha-2-adrenergic agonist to a peripheral nerve by peripheral nerve injection comprising:
a reservoir means for storing said agonist,
a therapeutically effective amount of said agonist disposed in said reservoir means,
a piston means movable along said reservoir means along a longitudinal axis for dispensing said agonist, and
a hollow needle mounted on said reservoir means for delivering said agonist to the peripheral nerve of a patient.

9. The device of claim 8, wherein the alpha-2-adrenergic agonist is clonidine.

10. The device of claim 8, wherein the alpha-2-adrenergic agonist is dexmedetomidine.

11. The device of claim 8, wherein the device is a syringe.

12. An injectable solution for delivering a therapeutic dose of an alpha-2-adrenergic agonist to a peripheral nerve by peripheral nerve injection comprising from 3μg to 1 mg of said alpha-2-adrenergic agonist.

13. The injectable solution of claim 12, comprising from 30 μg to 150 μg of said alpha-2-adrenergic agonist.

14. The injectable solution of claim 12, wherein the alpha-2-adrenergic agonist is clonidine.

15. The injectable solution of claim 12, wherein the alpha-2-adrenergic agonist is dexmedetomidine.

16. A method of treatment of neuropathic pain in a patient in need therof, comprising administering a therapeutic dose of an alpha-2-adrenergic agonist to a peripheral nerve by peripheral nerve injection every one to five weeks.

17. The method of claim 16, wherein said alpha-2-adrenergic agonist is clonidine.

18. The method of claim 16, wherein said alpha-2-adrenergic agonist is dexmedetomidine.
